(19) United States
(12) Patent Application Publication
(10) Pub. No.: US 2010/0190805 A1
(43) Pub. Date: Jul. 29, 2010

(54) PREPARATION FOR THE TREATMENT OF EQUINE LAMINITIS

(75) Inventors: Vincent Bachmann, Ohlstadt (DE); Ingo Lang, Ingelheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM VETMEDICA GMBH, Ingelheim (DE)

(21) Appl. No.: 12/601,329

(22) PCT Filed: Jun. 16, 2008
(86) PCT No.: PCT/DE2008/000979

§ 371 Date: Mar. 31, 2010

(30) Foreign Application Priority Data
Jun. 19, 2007 (DE) 10 2007 028 095.7

(51) Int. Cl. A61K 31/519; C07D 487/04; A61P 19/06

(52) U.S. Cl. 514/262.1; 544/262

(57) ABSTRACT

Disclosed is the use of active substances known from human medicine for treating gout in order to pharmaceutically treat equine laminitis, especially the use of a preparation containing at least allopurinol and/or hydrocortisone and/or powdered opium and/or prednisolone and/or prednisone.

The invention relates to a preparation for the treatment of laminitis, laminitis-associated pain and/or laminitis-associated inflammation in equids and particularly horses.

Founder or laminitis is a disease that occurs particularly in horses and denotes an aseptic inflammation of the hoof corium, i.e. an inflammation that is not caused by infective agents, in which the hoof capsule may become detached from the corium. Acute laminitis is an emergency and requires immediate treatment, while in extreme cases so-called “exungulation” may occur. Chronic laminitis may lead to rotation of the pedal bone.

The causes of laminitis have been investigated extensively and in some cases purely speculatively. Basically, the causes can be divided into two groups, namely mechanical trauma and toxic-chemical causes. What is common to all the causes of laminitis is that they lead to a disruption of the microcirculation of the blood in the region of the hoof corium. In mechanical traumatic laminitis, the stress laminitis is caused by overstressing of the hoof and is triggered in particular by long periods of running on hard ground or by overloading of one hoof, for example after the opposite leg has been rested. Long spells in the stable may also lead to laminitis on account of the resultant disruption of the blood circulation. In the case of the toxic-chemical causes, feed-induced laminitis is the commonest form of laminitis and is caused by incorrect feeding or the ingestion of toxic plants. Metabolic disorders are produced which may lead to an explosive multiplication of Streptococci in the large bowel and a massive release of lactic acid. This in turn leads to mass die-off of the bacteria that digest raw fibre and the release of endotoxins, leading to excessive acid levels throughout the body.

Where laminitis occurs, pathogenically this is an inflammation in the hoof in which there is a local disruption of the circulation, with the escape of lymph and blood corpuscles as solid components from the blood vessels of the laminae of the corium. This escape of fluid causes severe pain in the hoof as a result of the impossibility of expansion, while additionally the escape of fluid promotes the process of detachment of the laminae of the corium that engage frictionally with one another on the inside from the epidermal laminae on the outside.

In view of the fact that research has not fully clarified the causes, apart from the support measures provided by a farrier, treatment hitherto has consisted primarily of giving pain relief, using for example AC-promacin, heparin, gingko biloba and non-steroidal anti-inflammatories such as acetylsalicylic acid, for example. Additionally, detoxifying and kidney-stimulating substances as well as homoeopathic agents are often given as well.

Moreover, various homoeopathic and in some cases controversial healing treatments are known, such as bloodletting and the use of leeches.

To summarise, it can be stated that at present there are no truly effective means for successfully treating equine laminitis.

The present invention is based on the observation that there are clinically strong common factors and similarities between the human ailment gout and equine laminitis. In both laminitis and in gout the trigger may originate from the adrenal cortex and gonads. The two ailments, interestingly, are observed to be very complex metabolic disorders both in humans and in horses. However, there are pathogenically significant differences between gout and laminitis.

In addition the present invention is based on therapeutic successes achieved by the novel use of the preparations/medicament preparations described hereinafter.

The research carried out within the scope of the present invention has shown that a mixture of the most effective drugs known from human medicine is capable of combating the dangerous ailment of equine laminitis.

The invention therefore consists in the use of substances known from human medicine for the treatment of gout, for the drug treatment of laminitis in equids, particularly for treating horses.

For this purpose the use of a preparation containing at least allopurinol and/or cortisol and/or powdered opium and/or prednisolone and/or prednisone is particularly suitable.

Consequently, the present invention relates to the use of a preparation containing at least allopurinol and/or cortisol and/or powdered opium and/or prednisolone and/or prednisone for treating equine laminitis. Besides the use of the above-mentioned individual substances, or medicament preparations of the individual substances in question, the present invention relates in particular to the novel use of medicament preparations selected from among: allopurinol and cortisol; allopurinol and powdered opium; allopurinol and prednisolone; allopurinol and prednisone; cortisol and powdered opium; powdered opium and prednisolone; powdered opium and prednisone; prednisolone and prednisone; allopurinol, cortisol and powdered opium; allopurinol, cortisol and prednisolone; allopurinol, cortisol and prednisone; allopurinol, powdered opium and prednisolone; allopurinol, powdered opium and prednisone; allopurinol, prednisolone and prednisone; cortisol, powdered opium and prednisolone; cortisol, prednisolone and prednisone; powdered opium, prednisolone and prednisone.

This preparation provides an anti-inflammatory, anti-allergic, antirheumatic and immunosuppressant activity, so that the synthesis of triglycerides in horses can stabilise in the normal range.

The dosage of the substances known from human medicine is scaled up in accordance with a calculation based on the body weight of the animal in question.

According to a preferred embodiment the present invention relates to the use of a preparation containing allopurinol for treating laminitis in equids, particularly horses.

Allopurinol prevents the breakdown of purine into uric acid by inhibiting the enzyme xanthine oxidase and is therefore also referred to as a uricostatic. It brings about a lowering of the uric acid level in the blood, thereby enabling any deposits of uric acid in the tissues to be broken down and making it difficult for new deposits to form. The more frequently occurring precursors of uric acid (xanthines) can be excreted via the kidneys (ref. http://de.wikipedia.org/wiki/Allopurinol and http:www.vetpharm.uzh.ch/wir/00000031/5300_F.htm). Because of this mechanism of activity, allopurinol is used for treating gout in humans.

The use of allopurinol has also been described in horses, specifically for preventing reperfusion injury in colic (Allen, 1993). However, this indication differs fundamentally from laminitis. The activity mechanism postulated for preventing reperfusion injury is based on the mopping up of reactive oxygen radicals. Xanthine oxidase catalyses the conversion of hypoxanthine into xanthine and finally uric acid. During this reaction, oxygen radicals are released which have direct cytotoxic effects. These are thus inhibited by allopurinol and the active metabolite oxypurinol (cited in Mills et al., 1995).

Gout in humans is the depositing of uric acid crystals in the joints. As mentioned above, laminitis in horses is an aseptic inflammation of the hoof corium. It is therefore unexpected and surprising that allopurinol and other medicaments used for treating gout would have an effect on laminitis.

The pharmacokinetics of allopurinol in horses have been described (Mills et al., 1995). Allopurinol is rapidly converted into its active metabolite oxypurinol. The elimination half-life of allopurinol is 0.09 h, while that of oxypurinol is 1.09 h. The bioavailability of allopurinol after oral administration is poor (14.3%). However, even after oral administration of allopurinol in horses, the active substance is sufficiently systemically available, as the sum of the areas under the concentration/time curves of allopurinol and oxypurinol is identical to that of allopurinol administered intravenously. This indicates a high degree of absorption of allopurinol with subsequent metabolisation to form oxypurinol.

Another effect of the present invention relates to the surprising finding that the administration of allopurinol to equids, particularly horses, suffering from laminitis, leads to a fast relief of pain and reversal of the inflammation associated with laminitis. Consequently, in another aspect the present invention relates to the use of allopurinol for treating pain associated with laminitis, particularly in equids, such as horses, for example. In addition, the present invention relates to the use of allopurinol for treating inflammation associated with laminitis, particularly in equids, such as horses, for example.

Preparations containing allopurinol and/or cortisol and/or powdered opium and/or prednisolone and/or prednisone may be administered both orally and also by subcutaneous, intravenous or intramuscular route. The preferred methods of administration are oral or intravenous administration. Consequently, in another aspect, the present invention relates to oral, intravenous, subcutaneous or intramuscular preparations of allopurinol and/or cortisol and/or powdered opium and/or prednisolone and/or prednisone for treating laminitis in equids, preferably horses. Particularly preferred are corresponding allopurinol-containing preparations. The corresponding oral, intravenous, subcutaneous or intramuscular preparations may also be used, in another aspect of the present invention, for treating inflammation and/or pain associated with laminitis.

Allopurinol is particularly suitable for oral or intravenous use in a dosage of 1 to 50 mg/kg, preferably in a dosage of 2 to 20 mg/kg, more preferably in a dosage of 5 mg/kg per kg of bodyweight in equids. Consequently, in another aspect, the present invention relates to the use of a preparation containing allopurinol for treating laminitis, laminitis-associated inflammation and/or laminitis-associated pain in equids, produced in a dosage of 1 to 50 mg of allopurinol per kg of bodyweight of the equids. Preferably, corresponding allopurinol-containing preparations are produced for oral, subcutaneous, intravenous or intramuscular administration. The dosage mentioned here is preferably the dose to be administered per day.

The duration of treatment is measured according to the course of the disease. Generally, a treatment time of 1 to 10 days is effective. Preferably, the treatment is limited to 2 to 7 days, more preferably 3 to 4 days, preferably in the dosage mentioned above. Consequently, in another aspect, the present invention relates to the use of preparations of allopurinol and/or cortisol and/or powdered opium and/or prednisolone and/or prednisone for treating laminitis, laminitis-associated pain and/or laminitis-associated inflammation in equids, the corresponding preparation(s) being administered for 1 to 10 days, preferably 2 to 7 days, more preferably 3 to 4 days in a dosage as mentioned above.

The findings that underlie the invention will now be substantiated by means of Examples in the form of case reports and comparative blood tests on animals and humans.

### EXAMPLE 1

### Treatment of Horses Suffering from Laminitis with Allopurinol (1 to 50 mg/kg of Body Weight)

According to the present description of a number of applications, the oral administration of allopurinol to horses suffering from acute laminitis has the following effects: pain and inflammation decline after three days' treatment. The lameness is also reduced. The horses' appetite returns. Thus there are also positive effects on the general condition. Overall the condition of the horses is improved to the extent that they can be fitted with an orthopaedic hoof support roughly one week after the start of treatment. Only by treating with allopurinol is it possible to carry out this treatment as horses suffering acute pain cannot be fitted with supports.

### EXAMPLE 2

### Comparative Blood Tests

### Hoof:

problems in the distal locomotor apparatus
cellular investigation/laminae
toxicology
urology
neurological system
fructan/content, chemical structure/biology
gout/comparison with adrenocortex (activity)
A.C.T.H.
pigments
blood circulation
hypothalamus/interbrain
microcirculation
development of toxins in the metabolism/(general)
metabolic changes
situations of stress/shock
influence of the toxins on the blood circulation/thick, thin, etc. (evaluation)
structural elements/changes

### Blood Count/ACTH

glucocorticoids
cortisol/corticotrophin
lipoproteins fat metabolism+protein
pigmentation(s) (elements ppm) sodium urate
globinuria
myoglobinuria/urine
carbohydrate metabolism

### Metabolism

### Ionisation

electrolytes (in number)+(comparison)
potassium±+sodium+nitrate
T. lymphocytes
haemolytes/leukocytes
globulis/.R mega tetra plasma level
   .W
blood sugar level/glucose
   crystallisation
aldosterone/mineral corticoid
progesterone level
albumin/cholesterol
purine nitrate
   Fixing elements
   Non-fixing elements
   Browning of the skin

### Microcirculation

Dermis, epidermis

Laminitis Horse I

| | | | | Unit of | |
|---|---|---|---|---|---|
| Test | Result | Sign | Normal value | measurement | Remarks |
| Kidney: | | | | | 1) |
| Urea-N | 15 | | 10-20 | mg/dl | |
| creatinine | 1.28 | | <2.0 | mg/dl | |
| total protein | 6.7 | | 5.5-7.5 | g/dl | |
| sodium | 148 | | 125-150 | mmol/l | |
| chloride | 103 | | 95-105 | mmol/l | |
| potassium | 4.4 | | 2.8-4.5 | mmol/l | |
| inorg. phosphate | 0.83 | | 0.7-1.5 | mmol/l | |
| Liver: | | | | | |
| total bilirubin | 1.52 | | 0.5-3.1 | mg/dl | |
| ALT (GPT) | 16 | | 6-45 | U/l | |
| alk. phosphatase | 133 | | <338 | U/l | |
| y-GT | 21 | | <30 | U/l | |
| AST (GOT) | 408 | | 75-600 | U/l | |
| GLDH | 2.8 | | <12 | U/l | |
| albumin in the serum | 3.53 | | 2.5-4.5 | g/dl | |
| Pancreas: | | | | | |
| glucose | 89 | | 50-94 | mg/dl | |
| a-amylase | <10 | | <780 | U/l | |
| cholesterol | 107 | | 70-180 | mg/dl | |
| Muscle: | | | | | |
| CK | 215 | | <260 | U/l | |
| LDH | 470 | + | <400 | U/l | |
| calcium | 3.31 | | 2.3-3.4 | mmol/l | |
| magnesium | 0.67 | − | 0.7-0.9 | mmol/l | |
| total triglycerides | 53 | + | <50 | mg/dl | |
| Blood count: | | | | | |
| leukocytes | 7.9 | | 5-10 | G/l | |
| erythrocytes | 7.71 | | 6.0-12.0 | T/l | 2) |
| haemoglobin | 13.0 | | 11-17 | g/dl | |
| haematocrit | 33 | | 30-50 | % | |
| MCV | 43 | | 37-55 | fl | |
| HbE | 17 | | 13-19 | pg | |
| MCHC | 39 | + | 31-36 | g/dl | |
| thrombocytes | 119 | | 90-300 | F/l | |
| Differential blood | | | | | |
| count: | | | | | |
| basophilic gr. | 0 | | 0-2 | % | |
| eosinophilic gr. | 0 | | 0-4 | % | |
| band cells | 0 | | 0-6 | % | |
| segmented cells | 77 | + | 45-70 | % | |
| lymphocytes | 18 | − | 20-45 | % | |
| monocytes | 4 | | 0-5 | % | |
| basophilic gr. | 0 | | 0-150 | μl | |
| (absolute) | | | | | |
| eosinophilic gr. | 16 | − | 40-350 | μl | |
| (absolute) | | | | | |
| band cells | 0 | | 0-600 | μl | |
| (absolute) | | | | | |
| segmented cells | 6411 | | 3000-7000 | μl | |
| (absolute) | | | | | |
| lymphocytes (absolute) | 1448 | − | 1500-4000 | μl | |
| monocytes (absolute) | 272 | | 40-400 | μl | |
| atypical cells | 0 | | 0 | | |
| anisocytosis | 0 | | negative | | |
| polychromasia | 0 | | negative | | |

1) General note: When submitting whole blood please note: Glucose is no longer measured from whole blood. Fructosamine, potassium, LDH, phosphate and CPK values are falsely elevated when whole blood is stored for lengthy periods.
2) Erythrocytes haematocrit whole blood: 8.0-12.0 T/l 35-50% Warm blood: 6.5-9.0 T/l 33-45% Cold blood: 6.0-9.0 T/l 32-44% Pony: 5.5-8.5 T/l 30-40%

### Remarks:

Laminitis Horse II

| | | | | Unit of | |
|---|---|---|---|---|---|
| Test | Result | Sign | Normal value | measurement | Remarks |
| Major check-up | | | | | |
| Kidney: | | | | | 1) |
| Urea-N | 15.6 | | 10-20 | mg/dl | |
| creatinine | 1.3 | | <2.0 | mg/dl | |
| total protein | 7.0 | | 5.5-7.5 | g/dl | |
| sodium | 140 | | 125-150 | mmol/l | |
| potassium | 4.6 | + | 2.8-4.5 | mmol/l | |
| inorg. phosphate | 0.9 | | 0.7-1.5 | mmol/l | |
| Liver: | | | | | |
| total bilirubin | 1.21 | | 0.5-3.5 | mg/dl | |
| ALT (GPT) | 14.2 | | 2-15 | U/l | |
| alk. phosphatase | 211 | | <450 | U/l | |
| y-GT | 50 | + | <30 | U/l | |
| AST (GOT) | 417.0 | | 75-600 | U/l | |
| GLDH | 24 | + | <12 | U/l | |
| albumin in the serum | 3.00 | | 2.5-4.4 | g/dl | |
| Pancreas: | | | | | |
| glucose | 100 | + | 50-94 | mg/dl | |
| a-amylase | <10 | | <400 | U/l | |
| cholesterol | 85 | | 70-180 | mg/dl | |
| Muscle: | | | | | |
| CK | 206 | | <260 | U/l | |
| LDH | 561 | + | <400 | U/l | |
| calcium | 3.19 | | 2.3-3.4 | mmol/l | |
| magnesium | 0.63 | − | 0.7-0.9 | mmol/l | |
| total triglycerides | 68 | + | <50 | mg/dl | |
| Blood count: | | | | | |
| leukocytes | 6.6 | | 5-10 | G/l | |
| erythrocytes | 6.60 | | 6.0-12.0 | T/l | 1) |
| haemoglobin | 10.6 | − | 11-17 | g/dl | |
| haematocrit | 33 | | 30-50 | % | |
| MCV | 49 | | 37-55 | fl | |
| HbE | 16 | | 13-19 | pg | |
| MCHC | 32 | | 31-36 | g/dl | |
| thrombocytes | 98.9 | | 90-300 | G/l | |
| Differential blood | | | | | |
| count: | | | | | |
| basophilic gr. | 1 | | 0-2 | % | |
| eosinophilic gr. | 0 | | 0-4 | % | |
| segmented cells | 50 | | 45-70 | % | |
| lymphocytes | 41 | | 20-45 | % | |
| monocytes | 8 | + | 0-5 | % | |
| basophilic gr. | 66 | | | /μl | 2) |
| (absolute) | | | | | |
| eosinophilic gr. | 26 | −− | 40-350 | /μl | |
| (absolute) | | | | | |
| segmented cells | 3316 | | 3000-7000 | /μl | |
| (absolute) | | | | | |
| lymphocytes | 2665 | | 1500-4000 | /μl | |
| (absolute) | | | | | |
| monocytes | 491 | ++ | 40-400 | /μl | |
| (absolute) | | | | | |
| atypical cells | 0 | | 0 | | |
| anisocytosis | 0 | | negative | | |
| polychromasia | 0 | | negative | | |
| insulin | 67.0 | ++ | 7-51 | uU/ml | 3) |
| ACTH | 62.6 | | | pg/ml | 4) |

The material arrived in the laboratory already frozen.
Remarks:
1) Erythrocytes, haematocrit, whole blood: 8.0-12.0 T/l 35-50% Warm blood: 6.5-9.0 T/l 33-45% Cold blood: 6.0-9.0 T/l 32-44% Pony: 5.5-8.5 T/l 30-40%
2) Basophilic values up to 200 μl are regarded as physiological in the literature.
3) Partner laboratory
4) Reference range: Horse: 6.5-30.8 pg/ml Pony: 4.9-13.6 pg/ml
Values over 50 pg/ml should be regarded as suspicious.

Laminitis Horse III

| SEROLOGY | DIFFERENTIAL BLOOD COUNT |
|---|---|
| CK = 5.33 (−2.19) | band = % |
| LDH = 20.11 (−12.85) | segm = % |
| ALP = 3.28 (−8.89) | eos = % |
| TBIL = 21 (8.5-47.9) | mono = % |
| GOT = 4.57 (8.18) | lymph = % |
| GGT = 0.67 (−0.59) | baso = % |
| GPT = 0.17 (−0.46) | |
| BUN = 9.4 (3.34-6.68) | |
| CREA = 114 (−115) | LEUKOCYTES: |
| ALB = 30 (25-45) | |
| TPRO = 72 (55-75) | LEPTOSPIROSIS: |
| UA = 61 (54-65) | |
| CA = 3.69 (1.99-3.44) | BORELLIOSIS: |
| IP = 0.54 (0.81-1.45) | |
| MG = 0.77 (0.7-0.9) | ZN, SE |
| TG = (100-500) | |
| G1U = (3.05-5.00) | |

Human (gout)
Female
II

| | | | | | | standard |
|---|---|---|---|---|---|---|
| Analysis | name | result | +/− | unit | comment | range |
| HS | uric acid | 24.9 | ++ | mg/dl | | 2.3-6.1 |
| HAST | urea | 273 | + | mg/dl | | 10-50 |
| CREA | creatinine | 3.4 | + | mg/dl | | up to 1.1 |
| GLU | glucose/ | 176 | + | mg/dl | | 60-110 |
| | serum | | | | | |
| HBA1C | HbA1c | 6.0 | | % | | 4.1-6.2 |
| PHOS | inorg. | 4.3 | | mg/dl | | 2.6-4.5 |
| | phosphate | | | | | |
| CRP | quantitative | 64.2 | ++ | mg/l | | up to 5.0 |
| | CRP | | | | | |
| BBK | blood count | | | | | |
| | (small) | | | | | |
| LEUKO | leukocytes | 24.4 | ++ | /nl | | 4.0-10.5 |
| ERY | erythrocytes | 4.3 | | millions/ | | 3.5-5.4 |
| | | | | μl | | |
| HB | haemoglobin | 13.40 | | g/dl | | 12.0-16.0 |
| HK | haematocrit | 0.37 | | l/l | | 0.36-0.46 |
| MCV | MCV | 87 | | fl | | 81-99 |

Human (gout)
Male
III

| | | | | | | standard |
|---|---|---|---|---|---|---|
| Analysis | name | result | +/− | unit | comment | range |
| HS | uric acid | 10.4 | + | mg/dl | | 3.6-8.2 |
| CREA | creatinine | 1.7 | + | mg/dl | | up to 1.3 |
| GLU | glucose/serum | 93 | | mg/dl | | 60-110 |
| CHOL | cholesterol | 159 | | mg/dl | | up to |
| | | | | | | 200 . . . |
| TRIG | triglycerides | 105 | | mg/dl | | up to |
| | | | | | | 200 . . . |
| HDL | HDL | 43 | | mg/dl | | from 40 . . . |
| | cholesterol | | | | | |
| LDL | LDL | 101 | | mg/dl | | up to |
| | cholesterol | | | | | 160 . . . |
| LDLHDL | Art-Sider- | 2.4 | | kA | | up to 4.0 |
| | Index | | | | | |
| HBA1C | HbA1c | 7.8 | + | % | | 4.1-6.2 |
| K | potassium | 4.4 | | mmol/l | | 3.6-5.5 |
| LEUKO | leukocytes | 6.9 | | /nl | | 4.0-10.5 |
| ERY | erythrocytes | 4.6 | | millions/ | | 4.0-5.7 |
| | | | | μl | | |
| HB | haemoglobin | 13.5 | | g/dl | | 12.6-17.4 |
| HK | haematocrit | 0.39 | | l/l | | 0.39-0.52 |

Human (gout)
Female
IV

| | | | | | | standard |
|---|---|---|---|---|---|---|
| Analysis | name | result | +/− | unit | comment | range |
| HS | uric acid | 8.5 | + | mg/dl | | 3.6-8.2 |
| CREA | creatinine | 1.0 | | mg/dl | | up to 1.3 |
| GLU | glucose/serum | 76 | | mg/dl | | 60-110 |
| CHOL | cholesterol | 194 | | mg/dl | | up to |
| | | | | | | 200 . . . |
| TRIG | triglycerides | 249 | + | mg/dl | | up to |
| | | | | | | 200 . . . |
| HDL | HDL | 34 | − | mg/dl | | from 40 . . . |
| | cholesterol | | | | | |
| LDL | LDL | 120 | | mg/dl | | up to |
| | cholesterol | | | | | 160 . . . |
| LDLHDL | Art-Sider- | 3.5 | | kA | | up to 4.0 |
| | Index | | | | | |
| K | potassium | 4.2 | | mmol/l | | 3.6-5.5 |
| BBK | blood count | | | | | |
| | (small) | | | | | |
| LEUKO | leukocytes | 9.7 | | /nl | | 4.0-10.5 |
| ERY | erythrocytes | 4.9 | | millions/ | | 4.0-5.7 |
| | | | | μl | | |
| HB | haemoglobin | 15.0 | | g/dl | | 12.6-17.4 |
| HK | haematocrit | 0.44 | | l/l | | 0.39-0.52 |

Human (gout)
Male V

| | | | | | | standard |
|---|---|---|---|---|---|---|
| Analysis | name | result | +/− | unit | comment | range |
| HS | uric acid | 8.2 | | mg/dl | | 3.6-8.2 |
| CREA | creatinine | 1.1 | | mg/dl | | up to 1.3 |
| GLU | glucose/ | 35 | − | mg/dl | | 60-110 |
| | serum | | | | | |
| CHOL | cholesterol | 299 | + | mg/dl | | up to 200 |
| TRIG | triglycerides | 329 | + | mg/dl | | up to 200 |
| HDL | HDL | 45 | − | mg/dl | | from |
| | cholesterol | | | | | 30 . . . |
| LDL | LDL | 197 | ++ | mg/dl | | target |
| | cholesterol | | | | | values for |
| | | | | | | risk . . . |
| LDLHDL | Art-Sider- | 4.4 | + | kA | | up to 4.0 |
| | Index | | | | | |
| K | potassium | 6.6 | ++ | mmol/l | | 3.6-5.5 |
| TSH | TSH basal | 2.06 | | uU/ml | | 0.30-4.50 |
| BBK | blood count | | | | | |
| | (small) | | | | | |
| M0713A | practice | | | | | |
| | profile | | | | | |
| | 1 80713 | | | | | |

It should be pointed out that in cases where potassium has already crystallised in the hoof, no critical potassium values will be detectable in the blood count as potassium does not go back into the bloodstream. In such cases the veterinary surgeon will not be able to draw any conclusions from the potassium levels in the blood count.

Human (gout)
female I

| | | 17.04.07 | 10.04.07 | 05.04.07 | 02.04.07 | 29.03.07 | 24.03.07 |
|---|---|---|---|---|---|---|---|
| | | BNo. 1 L | BNo. 1 L | BNo. 3 L | BNo. 1 L | BNo. 1 L | BNo. 2 |
| BKS | ½ hr | | | 12/34 | | | |
| | 4/8 M M | | | | | | |
| | mmn.W. | | | | | | |
| CRP | <5.0 mg/l | | | | | | |
| BBK | | | — | — | — | | |
| QUICK | see | 49 | 32 | 25 | 62 | 86 | |
| | findings % | | | | | | |
| INR | ther. range | 1.60 | 2.10 | 2.60 | 1.40 | 1.20 | |
| | 2.0-4.5 | | | | | | |
| AP | 35-104 U/l | | | | | 70 | |
| BBG | | | | | | — | |
| GLU-F | 55-115 mg/dl | | | 178 | | 148 | |
| HbA1c | 4.1-6.2% | | | | | | |
| B1GUP | | | | | | — | |
| H8 | 2.3-6.1 mg/dl | | | | | 4.5 | |
| CREA | <1.1 mg/dl | | | | | 0.7 | |
| CKNAC | <167 U/l | | | | | | |
| LDH | 135-214 U/l | | | | | | |
| CHOL | see | | | | | 241 | |
| | findings | | | | | | |
| | mg/dl | | | | | | |
| TRIG | see | | | | | 135 | |
| | findings | | | | | | |
| | mg/dl | | | | | | |
| HDL | see | | | | | | |
| | findings | | | | | | |
| | mg/dl | | | | | | |
| LDL | see | | | | | | |
| | findings | | | | | | |
| | mg/dl | | | | | | |
| LDL-HDL | <4.0 | | | | | | |
| K | 3.6-5.5 mmol/l | | | | | 4.2 | |
| TSH | 0.30-4.50 uU/ml | | | | | | |
| AMYL | 28-100 U/l | | | | | | |
| LIPASE | <60 U/l | | | | | | |
| SERUM | | | | | | | |
| DIAPB | | | | — | | | |
| HBA | | | | | | | 6.6² |
| LDHISO | | | | | | | |
| LDH1 | 14.0-26.0% | | | | | | |
| LDH2 | 29.0-39.0% | | | | | | |
| LDH3 | 20.0-26.0% | | | | | | |
| LDH4 | 8.0-16.0% | | | | | | |
| LDH5 | 6.0-16.0% | | | | | | |
| LEUKO | 4.0-10.5/ | | 6.9 | 5.6 | 5.3 | 5.4 | |
| | nl | | | | | | |
| ERY | 3.5-5.4 mil/μl | | 3.2 | 3.1 | 3.0 | 3.0 | |
| HB | 12.0-16.0 g/dl | | 9.7 | 9.4 | 8.8 | 9.0 | |
| HK | 0.36-0.46 l/l | | 0.31 | 0.30 | 0.29 | 0.28 | |
| MCV | 81-99 fl | | 97 | 98 | 98 | 96 | |
| MCH | 28-34 pg | | 30 | 30 | 30 | 30 | |
| MCHC | 32-36 g/dl | | 31 | 31 | 30 | 32 | |
| THROMB | 130-430/ | | 407 | 400 | 373 | 340 | |
| | nl | | | | | | |
| FE | 49-151 μg/dl | | | 116 | 181 | 101 | |
| FERRI | 20-291 μg/l | | | 26 | | | |
| GGT | <42 U/dl | | | | | 33 | |
| GPT | <35 U/l | | | | | 16 | |
| DIFB | | | | | | — | |
| NEUT | 45-75% | | | | | 84 | |
| LYMPHO | 25-45% | | | | | 11 | |
| MONO | <14% | | | | | 5 | |
| EOS | <7% | | | | | 0 | |
| BASO | <1% | | | | | 0 | |
| SIZE | | | | | | | 164² |
| WEIGHT | | | | | | | 64² |
| RR_SYST | | | | | | | 110² |
| RR_DIAS | | | | | | | 85² |
| PULSE | | | | | | | 74² |
| BLOOD | MG/DL | | | | | | 210² |
| SUGAR | mg/dl | | | | | | |
| HI RISK | | | | | | | only at² |
| CHOLESTEROL | MG/DL | | | | | | |
| | mg/dl | | | | | | |
| TRIGLYCERIDES | MG/DL | | | | | | |
| | mg/dl | | | | | | |
| | | 29.01.07 | 09.01.07 | 14.12.06 | 13.12.06 | 14.11.06 | 23.10.06 |
| | | BNo. 2 L | BNo. 2 L | BNo. 3 L | BNo. 1 L | BNo. 2 L | BNo. 1 L |
| BKS | ½ hr | 60/66 | 50/59 | | | 52/66 | |
| | 4/8 M M | | | | | | |
| | mmn.W. | | | | | | |
| CRP | <5.0 mg/l | | | | | 2.4 | 14.5 |
| BBK | | | | | | | |
| QUICK | see | 24 | 25 | | 13 | 28 | 20 |
| | findings % | | | | | | |
| INR | ther. range | 2.60 | 2.50 | | 3.90 | 2.40 | 3.00 |
| | 2.0-4.5 | | | | | | |
| AP | 35-104 U/l | | 57 | | 57 | | 62 |
| BBG | | | — | | — | | — |
| GLU-F | 55-115 mg/dl | 210 | 235 | | 232 | 156 | 142 |
| HbA1c | 4.1-6.2% | 6.6 | 7.0 | | 6.7 | | 7.0 |
| B1GUP | | | — | | — | | — |
| H8 | 2.3-6.1 mg/dl | | 10.0 | | 9.5 | | 10.1 |
| CREA | <1.1 mg/dl | 0.8 | 0.7 | | 0.8 | | 0.9 |
| CKNAC | <167 U/l | | | | 65 | | 118 |
| LDH | 135-214 U/l | | | 492 | 463 | 500 | 506 |
| CHOL | see | 252 | 274 | | 252 | | 237 |
| | findings | | | | | | |
| | mg/dl | | | | | | |
| TRIG | see | 157 | 200 | | 181 | | 133 |
| | findings | | | | | | |
| | mg/dl | | | | | | |
| HDL | see | 58 | 57 | 58² | 58 | | 45 |
| | findings | | | | | | |
| | mg/dl | | | | | | |
| LDL | see | 190 | 198 | 188² | 188 | | 174 |
| | findings | | | | | | |
| | mg/dl | | | | | | |
| LDL-HDL | <4.0 | 3.3 | 3.5 | | 3.3 | | 3.8 |
| K | 3.6-5.5 mmol/l | | 3.2 | | 3.7 | | 3.8 |
| TSH | 0.30-4.50 uU/ml | | | | | | 1.39 |
| AMYL | 28-100 U/l | | | | 75 | | 81 |
| LIPASE | <60 U/l | | | | 30 | | 72 |
| SERUM | | | | — | | | |
| DIAPB | | — | — | | — | | — |
| HBA | | | | 6.7² | | | |
| LDHISO | | | | — | | | |
| LDH1 | 14.0-26.0% | | | 36.5 | | | |
| LDH2 | 29.0-39.0% | | | 40.0 | | | |
| LDH3 | 20.0-26.0% | | | 14.3 | | | |
| LDH4 | 8.0-16.0% | | | 4.8 | | | |
| LDH5 | 6.0-16.0% | | | 4.4 | | | |
| LEUKO | 4.0-10.5/ | | 6.7 | | 4.6 | | 7.1 |
| | nl | | | | | | |
| ERY | 3.5-5.4 mil/μl | | 3.8 | | 3.8 | | 3.9 |
| HB | 12.0-16.0 g/dl | | 11.9 | | 12.1 | | 12.4 |
| HK | 0.36-0.46 l/l | | 0.37 | | 0.37 | | 0.38 |
| MCV | 81-99 fl | | 97 | | 95 | | 98 |
| MCH | 28-34 pg | | 31 | | 32 | | 32 |
| MCHC | 32-36 g/dl | | 32 | | 33 | | 33 |
| THROMB | 130-430/ | | 241 | | 233 | | 337 |
| | nl | | | | | | |
| FE | 49-151 μg/dl | | 91 | | 120 | | 71 |
| FERRI | 20-291 μg/l | | | | | | |
| GGT | <42 U/dl | | 37 | | 38 | | 38 |
| GPT | <35 U/l | | 22 | | 24 | | 22 |
| DIFB | | | — | | — | | — |
| NEUT | 45-75% | | 84 | | 81 | | 84 |
| LYMPHO | 25-45% | | 11 | | 15 | | 9 |
| MONO | <14% | | 4 | | 4 | | 6 |
| EOS | <7% | | 1 | | 1 | | 1 |
| BASO | <1% | | 0 | | 0 | | 1 |
| SIZE | | | | 164² | | | |
| WEIGHT | | | | 64² | | | |
| RR_SYST | | | | 85² | | | |
| RR_DIAS | | | | | | | |
| PULSE | | | | | | | |
| BLOOD | MG/DL | | | 232² | | | |
| SUGAR | mg/dl | | | | | | |
| HI RISK | | | | only at² | | | |
| CHOLESTEROL | MG/DL | | | 252² | | | |
| | mg/dl | | | | | | |
| TRIGLYCERIDES | MG/DL | | | 181² | | | |
| | mg/dl | | | | | | |

### REFERENCES

Allen D G, Pringle J K & Smith D:

### Handbook of Veterinary Drugs.

JB Lippincott Company, Philadelphia (USA); 678 pp, 1993

### ISBN: 0-397-51265-1

Mills P C, Dunnett M and Smith N C:

The pharmacokinetics of oral and intravenous allopurinol and intravenous oxypurinol in the horse. J Vet Pharmacol Ther 18(6): 451-456, 1995

1. Medicament preparation comprising an active substance known from human medicine for the treatment of gout, for the drug treatment of laminitis, laminitis-associated inflammation and/or laminitis-associated pain in equids.

2. Medicament preparation according to claim 1, wherein said medicament preparation comprises an active substance selected from the group consisting of allopurinol, cortisol, powdered opium, prednisolone, prednisone, and mixtures thereof.

3. Medicament preparation according to claim 1, wherein said medicament preparation is for oral, intravenous, subcutaneous or intramuscular administration.

4. Medicament preparation according to claim 1, wherein said equids are horses.

5. Medicament preparation according to claim 1, wherein said medicament preparation comprises allopurinol.

6. Medicament preparation according to claim 5, wherein said medicament preparation is administered in a dosage of 1 to 50 mg/kg of body weight of the equids.

7. Medicament preparation according to claim 5, wherein said medicament preparation is administered for 1 to 10 days.

8. Use of a medicament preparation according to claim 1 for the treatment of laminitis, laminitis-associated pain and/or laminitis-associated inflammation in equids.

9. Method of treating laminitis, laminitis-associated pain and/or laminitis-associated inflammation in equids, comprising administering a medicament preparation according claim 1.
